(19) United States
(12) Patent Application Publication
(10) Pub. No.: US 2014/0243684 A1
(43) Pub. Date: Aug. 28, 2014

(54) SYSTEM AND METHOD FOR CREATING, PROCESSING, AND DISPLAYING TOTAL BODY IMAGE

(71) Applicants: Clara Curiel, Tucson, AZ (US); Karleen Seybold, Tucson, AZ (US); Alex Diaz, San Luis Obispo, CA (US); Scott Tyo, Tucson, AZ (US); Andrey Alenin, Tucson, AZ (US)

(72) Inventors: Clara Curiel, Tucson, AZ (US); Karleen Seybold, Tucson, AZ (US); Alex Diaz, San Luis Obispo, CA (US); Scott Tyo, Tucson, AZ (US); Andrey Alenin, Tucson, AZ (US)

(73) Assignee: DermSpectra LLC, Tucson, AZ (US)

(21) Appl. No.: 13/781,735

(22) Filed: Feb. 28, 2013

Related U.S. Application Data
(63) Continuation-in-part of application No. 13/778,942, filed on Feb. 27, 2013.

(51) Int. Cl. A61B 5/00

(52) U.S. Cl. CPC A61B 5/0077; A61B 5/0013; A61B 5/7425; A61B 5/7475; A61B 5/444
USPC 600/476

(57) ABSTRACT

A system and method for creating and processing a total body image of a user from a plurality of user images. The system and method also includes enabling a medical professional/provider to access the user images and manage medical care for the user.

### CROSS-REFERENCE TO RELATED APPLICATIONS

This application claims priority to patent application having Ser. No. 13/778,942 filed Feb. 27, 2013, which is herein incorporated by reference in its entirety.

### FIELD OF INVENTION

The present invention generally relates to a system and method for creating, processing, and displaying a total body image. More particularly, the present invention relates to a system and method for automatically creating, processing, and displaying a total body image of a user/patient after capturing a plurality of body images of the user/patient. The plurality of body images used to create the total body image may be taken using an automated method and/or apparatus that requires minimal assistance from medical staff. The system and method for creating, processing, and displaying a total body image includes a graphic user interface for a technician/medical assistant which aids in the capture of a plurality of the user's/patient's body images, a program application for stitching the plurality of body images together to form a user/patient total body image, a program application that enables a medical provider/professional to obtain access to the user/patient total body image, and a graphic user interface for the medical provider/professional that enables the medical provider/professional to view user/patient body images and user/patient data and information, and to manage user/patient medical care.

### BACKGROUND OF THE INVENTION

Medical care has become increasingly costly while at the same time resulting in decreased time for a user/patient to interface with medical professionals/providers. In addition, users/patients are often treated by a number of different medical professionals/providers at a number of different sites or locations throughout their lifetimes.

Most medical professionals/providers use electronic medical records, which are computerized medical records, to document user/patient information and medical care. Electronic medical records allow for storage, retrieval, and modification of user/patient information and medical care. An electronic medical record can be used to read and write a user's/patient's medical record through a workstation and, in some systems and health care settings, electronic medical records can even be used to read and write a user/patient's medical record through mobile devices that are handwriting capable. In addition, some electronic medical record systems can automatically monitor clinical events by analyzing user/patient data for an electronic health/medical record to predict, detect and potentially prevent adverse events.

Digital Imaging and Communications in Medicine (DICOM) is a standard for handling, storing, printing, and transmitting information in medical imaging that includes a file format definition and a network communications protocol. The communication protocol uses TCP/IP to communicate between systems. DICOM has been widely adopted by hospitals and enables the integration of scanners, servers, workstations, printers, and network hardware from multiple manufacturers into a picture archiving and communication system. Different devices come with DICOM conformance statements which clearly state which DICOM classes they support. DICOM files can be exchanged between two entities that are capable of receiving image and patient data in DICOM format.

Monitoring of a patient's skin is typically performed by a medical professional/provider who observes the skin and then takes notes reflecting their observations. Sometimes, the medical professional/provider will also take a photograph of suspicious areas of a patient's skin to enable further evaluation of the area at a later date. However, this type of monitoring is not as accurate and reliable as consistent and total body imaging of a patient's skin. DICOM already provides a format in which medical imaging of the skin may be stored, printed, and transmitted between entities and/or medical professionals/providers. Accordingly, there is a need for a system and method for creating, processing, and displaying a total body image that is automated, consistent, reliable and able to integrate the DICOM format so that medical professionals/providers can easily access accurate and reliable skin imaging information relating to their patients.

### SUMMARY OF THE INVENTION

The present invention is directed toward a system and method for creating and processing, a total body image, as well as displaying that total body image. In one exemplary embodiment, the system for creating and processing a total body image includes a device for capturing a plurality of overlapping images of a user/patient, a server for storing and transmitting user images and user information, and a processor in communication with a program application for stitching together the plurality of overlapping images of the user to create a total body image of the user/patient. In another exemplary embodiment, the system for creating and processing, and further displaying a total body image includes a device for capturing a plurality of overlapping images of a user/patient, a server for storing and transmitting user images and user information, a processor in communication with a program application for stitching together the plurality of overlapping images of the user to create a total body image of the user/patient, and at least one computer and/or mobile computing device (such as a laptop, an electronic tablet, an electronic notebook, etc.) and a program application for a graphic user interface that enables a medical professional/provider to access user/patient images, including a user/patient total body image, and manage the medical care of the user/patient.

The medical professional/provider may access the user/patient information and images form a wired device or a wireless device. The server included in the system is capable of storing and transmitting user/patient images and user/patient information between entities.

The device for capturing a plurality of overlapping images of a user/patient may be automated and it may also include an auto focus algorithm that automatically adjusts the focus of one or more cameras for different user/patient poses based on a desired body focus feature.

In yet another exemplary embodiment of the present invention, the system and method for creating, processing, and displaying a total body image may further include a network in communication with the server and it may still further include one or more external databases in communication with the server via the network.

The system of the present invention for creating, processing, and displaying a total body image may also include a program application that enables a medical professional/provider to measure and mark skin abnormalities (such as skin lesions, etc.) with a labeling tool, record text and/or verbal observations of the skin and/or skin abnormalities, compare user/patient images and user/patient observations and data to previous same area user/patient images and user/patient observations and data taken and recorded at different dates/times (e.g. side by side comparisons), record diagnoses, record and schedule tests to be performed, create alerts for skin abnormalities that need follow up, record treatment plans and case management, and apply ICD (International Statistical Classification of Diseases and Related Health Problems) codes and CPT (Current Procedural Terminology) codes when diagnosing and assessing using the graphic user interface for medical professionals/providers.

The present invention is also directed to a method for creating and processing a total body image which includes the steps of capturing a plurality of overlapping images of a user and stitching together the plurality of overlapping images of the user to create a total body image of the user. The total body image of the user may be a two dimensional or three dimensional image. In one exemplary embodiment, the step of capturing the overlapping images may utilize an automated imaging station/device. In addition, that embodiment of the method of the present invention for creating and processing a total body image may also include the step of providing a graphic user interface for a technician/medical assistant that enables the technician/medical assistant to control one or more aspects of capturing the overlapping images of the user utilizing the automated imaging station/device. In addition, the method may also include the step of utilizing an automatic focusing algorithm to automate focusing of cameras within the imaging station/device by determining an area of interest for each camera in each of one or more predetermined pose& undertaken by the user when utilizing the automated imaging station/device.

In addition to a method for creating and processing a total body image, the present invention is also directed to a method for creating, processing and displaying a total body image which includes all of the above mentioned steps relating to creating and processing a total body image in addition to the step of accessing a server to store and transmit user/patient images and user/patient information and the step of transmitting the user/patient images and user/patient information to at least one computer and/or mobile computing device by utilizing a graphic user interface that enables a medical professional/provider to access the user/patient images and manage medical care for the user/patient. In addition, the method of the present invention for creating, processing, and displaying a total body image may also include the ability to enable the medical professional/provider that accesses a user's total body image to focus on and magnify areas within the user's total body image that are selected by the medical professional/provider. The method of the present invention may also enable a medical professional/provider, via the graphic user interface, to measure and mark skin abnormalities (such as skin lesions, etc.) with labeling tool, record text and/or verbal observations of the skin and/or skin abnormalities, compare user/patient images and user/patient observations and data to previous same area user/patient images and user/patient observations and data taken and recorded at different dates/times (e.g. side by side comparisons), record diagnoses, record and schedule tests to be performed, create alerts for skin abnormalities that need follow up, record treatment plans and case management, and apply ICD (International Statistical Classification of Diseases and Related Health Problems) codes and CPT (Current Procedural Terminology) codes when diagnosing and assessing using the graphic user interface for medical professionals/providers.

### BRIEF DESCRIPTION OF THE DRAWINGS

The figures illustrate various embodiments of the present invention by way of example, and not by way of limitation. Embodiments of the present invention may include part or all of the features shown in one of these figures, or may include features from two or more figures. Embodiments of the present invention may also include features described in the specification, or elements of features described in the specification. Furthermore, embodiments of the present invention may include features that would be familiar to a person of ordinary skill in the art having studied this document. Thus, a more complete understanding of the present invention may be derived by referring to the detailed description and claims when considered in connection with the drawing figures where like reference numbers refer to similar elements throughout the figures.

FIG. 1 is a block diagram of an exemplary embodiment of a system for creating, processing, and displaying a total body image of a user in accordance with the present invention.

FIG. 2 is an outer perspective view of an exemplary embodiment of an imaging station/device for use in accordance with the system for creating, processing, and displaying a total body image of the present invention.

FIG. 3 is a top plan view showing the interior of the exemplary embodiment of the imaging station/device shown in FIG. 2

FIG. 4 is an outer perspective view of the exemplary embodiment of the imaging station/device shown in FIGS. 2 and 3.

FIG. 5 is an exemplary software block diagram showing connections to a computer located within the imaging station/device that is included in the system for creating, processing, and displaying a total body image of a user.

FIG. 6 is an exemplary screen shot of a graphical user interface for a technician/medical assistant when a technician/medical assistant is involved in operating the imaging station/device that is included in the system for creating, processing, and displaying a total body image of a user.

FIG. 7 is an exemplary software block diagram showing connections between the imaging station/device and other devices and interfaces that are included in the system for creating, processing, and displaying a total body image of a user.

FIG. 8 is a schematic showing an exemplary process of how the plurality of overlapping images of a user are stitched together to form a total body image of the user within the system for creating, processing, and displaying a total body image of a user.

FIG. 9 is a flowchart-like schematic showing an exemplary method for creating, processing, and displaying a total body image of a user.

FIGS. 10-27 show exemplary frames/screen shots of the graphic user interface for medical professionals/providers used with the system and method for creating and processing a total body image of a user in accordance with the present invention.

### DETAILED DESCRIPTION

The present invention is directed to a system and method for creating and processing a total body image where the system includes a device for capturing a plurality of overlapping images of a user/patient, a server for storing and transmitting user/patient images and user/patient information, and a processor in communication with a program application for stitching together the plurality of overlapping images of the user/patient to create a total body image of the user/patient. The present invention is also directed to a system and method for creating, processing, and displaying a total body image where the system includes a device for capturing a plurality of overlapping images of a user/patient, a server for storing and transmitting user/patient images and user/patient information, a processor in communication with a program application for stitching together the plurality of overlapping images of the user/patient to create a total body image of the user/patient, and the ability to interface with a computer, electronic tablet, electronic notebook, or any other mobile computing device of a medical professional/provider through a program application that provides a graphic user interface that enables the medical professional/provider to access user/patient images and information and manage medical care for the user/patient.

The system and method for creating a total body image of the present invention creates and processes a total body image of a user/patient after capturing a plurality of body images of the user/patient. The plurality of body images used to create the total body image may be captured by using an automated method and/or apparatus that requires minimal assistance from medical staff/personnel. Examples of such a method and apparatus can be seen in U.S. Patent Application having Ser. No. 13/778,942 which is not meant to be limiting with respect to the system and method of the present invention. The system and method of creating, processing, and displaying a total body image of the present invention also includes a graphic user interface for a technician/medical assistant which enables the technician/medical assistant to manually or automatically control capture of images of a user/patient with the imaging station/device, a program application that enables a medical provider/professional to obtain access to the user/patient total body image, and a graphic user interface for the medical provider/professional that enables the medical provider/professional measure and mark skin abnormalities (such as skin lesions, etc.) with a labeling tool, record text and/or verbal observations of the skin and/or skin abnormalities, compare user/patient images and user/patient observations and data to previous same area user/patient images and user/patient observations and data taken and recorded at different dates/times (e.g. side by side comparisons), record diagnoses, record and schedule tests to be performed, create alerts for skin abnormalities that need follow up, record treatment plans and case management, and apply ICD (International Statistical Classification of Diseases and Related Health Problems) codes and CPT (Current Procedural Terminology) codes when diagnosing and assessing using the graphic user interface for medical professionals/providers.

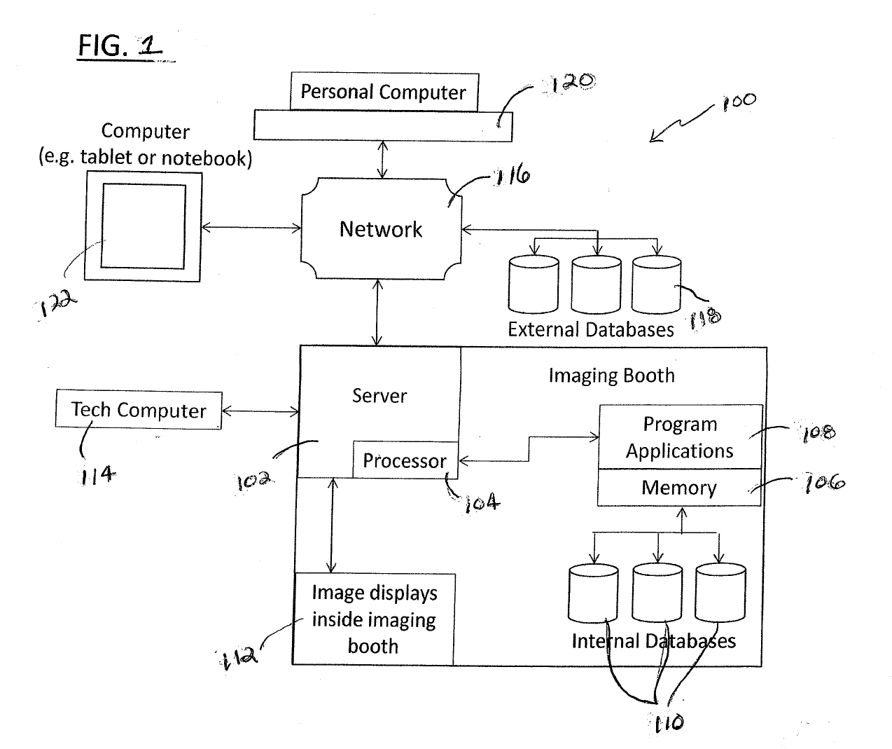

FIG. 1 is a block diagram of an exemplary embodiment of a system for creating, processing, and displaying a total body image of a user in accordance with the present invention. System 100 may include a server 102 having a processor 104 which is in communication with a memory 106 having one or more program applications 108 stored therein such as, for example, a Body Imaging Program which controls the movement and function of the cameras in the imaging station/booth and the instructions to the user or patient and an Audio and/or Visual Program that provides verbal and/or video instructions and posing guides to the patient in order to demonstrate the correct poses to the patient. Memory 106 may also be in communication with internal databases 110 that can include, but are not limited to, parameters and standards for assessing skin neoplasms and/or skin variations and/or previous images taken of the patient for comparison to current images. The server 102 may also be in communication with the image/video displays 112 that are positioned and mounted within the interior of the imaging station/booth. Server 102 may also be in communication with a computer 114 that is for the technician or medical assistant so that the technician or medical assistant can intervene if necessary to control certain portions of the program applications or to make necessary selections required by the program applications.

The server 102, processor 104, memory 106, program applications 108, internal databases 110, and image/video displays or monitors 112 may all constitute hardware and/or software that are located within a portion of the imaging station/device. The server 102 may further be in communication with a network 116 that can access external databases 118 which may also include, but are not limited to, parameters and standards for assessing skin neoplasms and/or skin variations and/or previous images taken of the patient for comparison to current images for any number of the previous described applications for the automated total body imaging system of the present invention. In addition, it should be understood that the total body imaging system of the present invention can easily interface with any type of electronic medical record (EMR) systems, particularly those using a DICOM format. Server 102 can also provide access to the program applications as well as information and data produced from the program applications to personal computers 120 and personal notebooks and/or tablets 122 or any other mobile computing device of physicians or medical providers via network 116 so that physicians and/or medical providers who are responsible for viewing and assessing the total body images do not have to be present at the imaging station or imaging station/booth while imaging is taking place.

Program application(s) that are in communication with server 102 may include one or more of: i) a program application for taking, capturing, and storing the overlapping images produced by cameras contained within the imaging station/device, ii) an automatic focusing algorithm to automate focusing of cameras within the imaging station/device by determining an area of interest for each camera in each of one or more predetermined poses undertaken by a user, iii) a program that functions to stitch together the overlapping images taken by the cameras contained within the imaging station/device to create a total body image, iv) a program application that enables a medical professional and/or medical facility (such as hospitals, medical clinics, etc.) to obtain wireless access to the overlapping images and total body image in order to view the overlapping images and total body image, compare a plurality of the overlapping images and total body image of a same user/patient taken at different times (i.e. side by side comparisons), the ability to zoom in and focus on specific areas of the user's/patient's body when viewing the plurality of images and/or total body image of the user/patient, measure and mark skin abnormalities (such as skin lesions, etc.) with a labeling tool, record text and/or verbal observations of the skin and/or skin abnormalities, record diagnoses, record and schedule tests to be performed, create alerts for skin abnormalities that need follow up, record treatment plans and case management, apply ICD (International Statistical Classification of Diseases and Related Health Problems) codes and CPT (Current Procedural Terminology) codes when diagnosing and assessing using the graphic user interface for medical professionals/providers, create electronic medical records that include the user/patient images, and/or send the user/patient images and related notes and/or data to another medical professional and/or medical facility, and v) a program application that enables a medical professional and/or medical facility having access to the images of a user/patient to interface with other existing electronic medical record databases from other medical professionals and/or medical facilities utilized by the user so that the images of the user/patient can be compared to other existing electronic record databases.

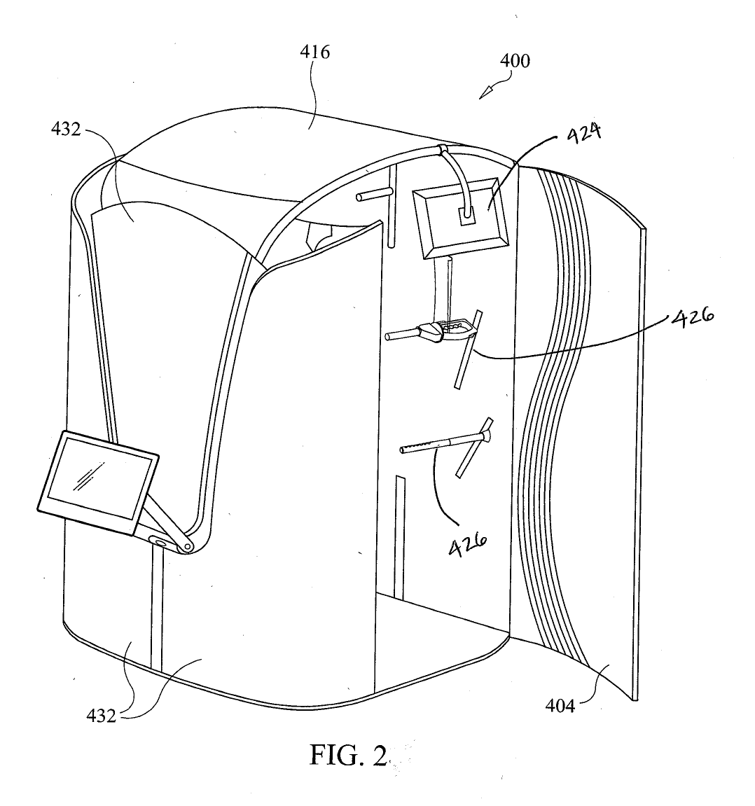

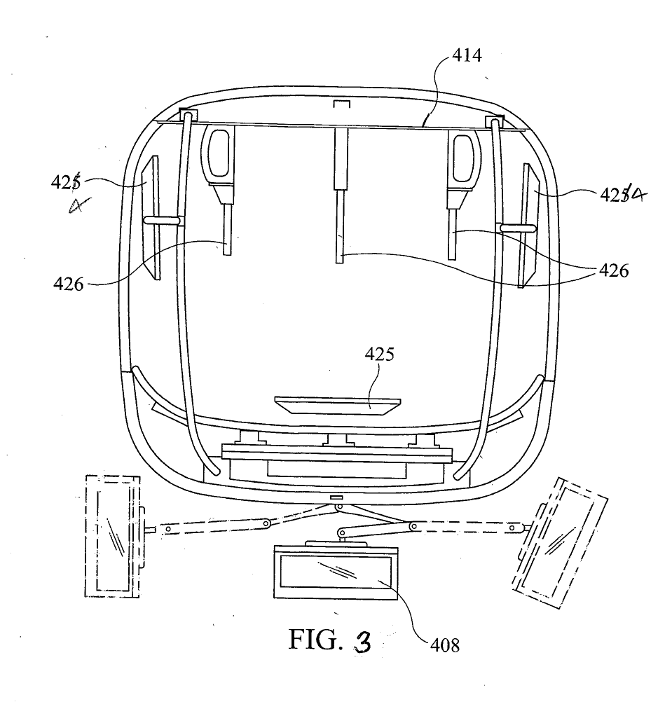

An outer perspective view of an exemplary embodiment of an imaging station/device for use in accordance with the system for creating, processing, and displaying a total body image of the present invention is shown in FIG. 2 and a top plan view showing the interior of the exemplary embodiment of the same imaging station/device is shown in FIG. 3. As previously described in application having Ser. No. 13/778,942, imaging station/device 400 can include a top panel 416, a rear panel 414, a side panel 404 that functions as a door or has a door contained within it, and moveable front panels 432 which can be moved to access cameras, computers, and other hardware contained within the imaging station/ device 400. Imaging station/device 400 also includes body positioning members 426 that assist a user/patient to correctly assume various body poses for user/patient body images, video displays 424 for enabling a user/patient to see posing instructions, and a technician computer device 408 that may be adjustably connected to an outer surface of the imaging station/booth 400 to enable a technician to control the program applications associated with the use of the imaging station/booth 400.

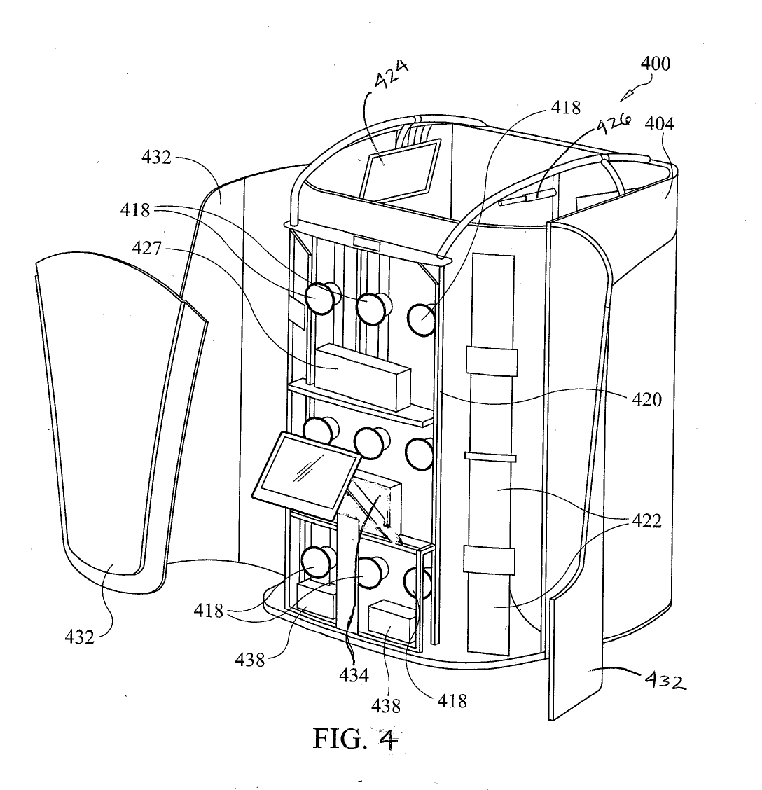

FIG. 4 is an outer perspective view of the exemplary embodiment of the imaging station/device shown in FIGS. 2 and 3. Outer front panels 432 of imaging station/device 400 are shown in an open position. As shown, the outer front panels 432 can be moved and/or removed to provide access to the plurality of cameras 418 secured to the frame 420, one or more speaker components 427, light panels 422, a computer processing unit 434 in communication with one or more program applications relating to the use of the imaging station/booth 400, and other electronic components 438 and connections relating to the operation of the imaging station/booth 400. Examples of the program applications include those previously set out above.

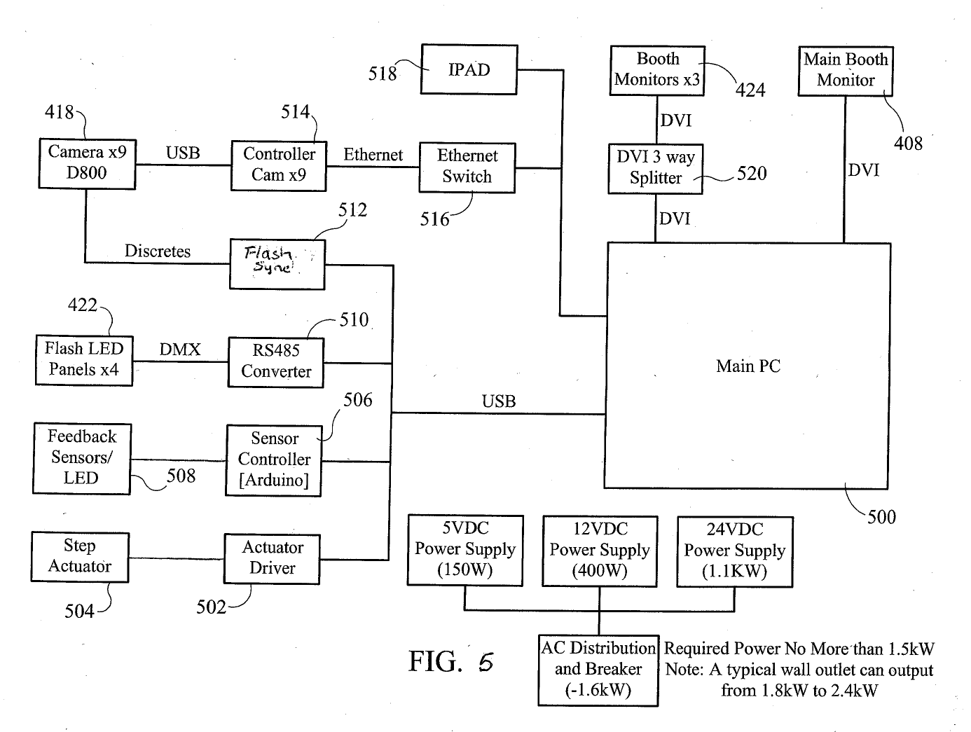

FIG. 5 is an exemplary software block diagram showing connections to a computer located within the imaging station/device that is included in the system for creating, processing, and displaying a total body image of a user. The electrical layout includes a Main PC 500 which is in turn connected to i) an actuator driver 502 that is in turn connected to a step actuator 504 for a moveable step/foot plate contained within an imaging station/booth to assist a user/patient in exhibiting various predetermined poses, ii) a sensor controller 506 that is in turn connected to feedback sensor 508 for the LEDs contained within body positioning members 426 that are capable of being lit when properly engaged by a user/patient, iii) a converter 510 which is in turn connected to the light panels 422 contained within the booth which function to provide proper lighting for capturing images using cameras 418, and iv) a flash sync 512 that is in turn connected to cameras 418 which are in turn each connected to a controller 514 which are all in turn connected to an Ethernet switch 516 which itself enables connectivity to the Main PC 500 and tablet and notebook devices such as an IPAD 518, or any other mobile computing devices. The Main PC 500 is also connected to the main station/booth monitor (or tech computer and monitor 408) as well as a 3 way splitter 520 which is in turn connected to each of the visual display devices 424. This is just one exemplary embodiment of how the components in an imaging station/device are connected in order to provide the system and method of the present invention for creating, processing and displaying a total body image of a user/patient.

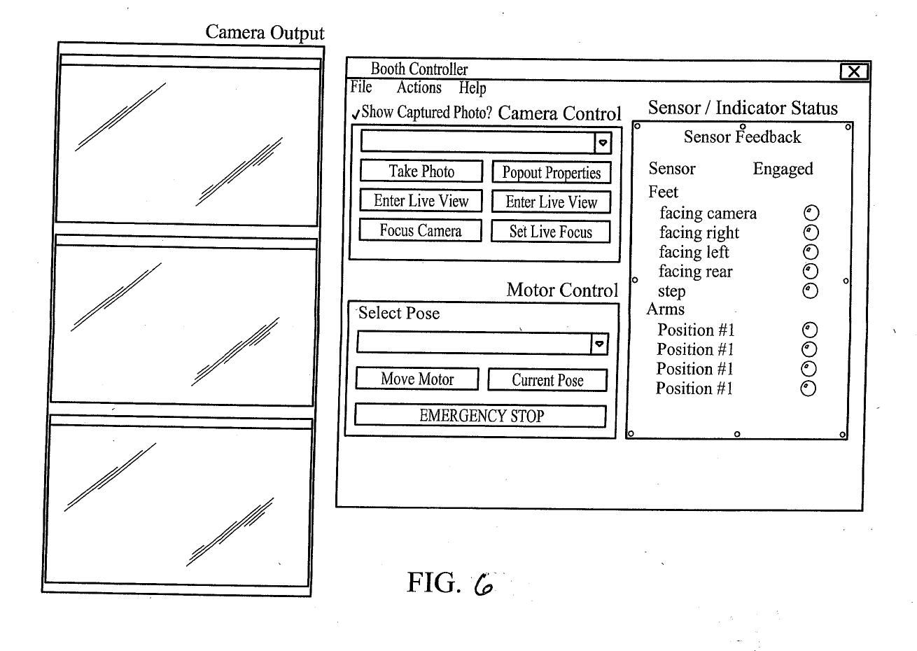

An exemplary screen shot of a graphical user interface for a technician/medical assistant when a technician/medical assistant is involved in operating the imaging station/device that is included in the system for creating, processing, and displaying, a total body image of a user/patient is shown in FIG. 6. This is just one exemplary screen shot of the graphic user interface that can be accessed by a technician/medical assistant in conjunction with the system and method for creating, processing, and displaying a total body image in accordance with the present invention. As can be seen in FIG. 6, the technician/medical assistant may select specific poses for the user/patient to undertake as part of the imaging process, focus and/or take the photos taken as part of the imaging process, and/or view the user's/patient's proper engagement with various sensors in communication with the body posing elements contained in the imaging station/device to ensure that the patient has undertaken the correct pose for taking images. These are just some examples of the components that a technician/medical assistant may be able to control via a graphic interface for the technician/medical assistant as part of the system and method of the present invention for creating, processing, and displaying a total body image.

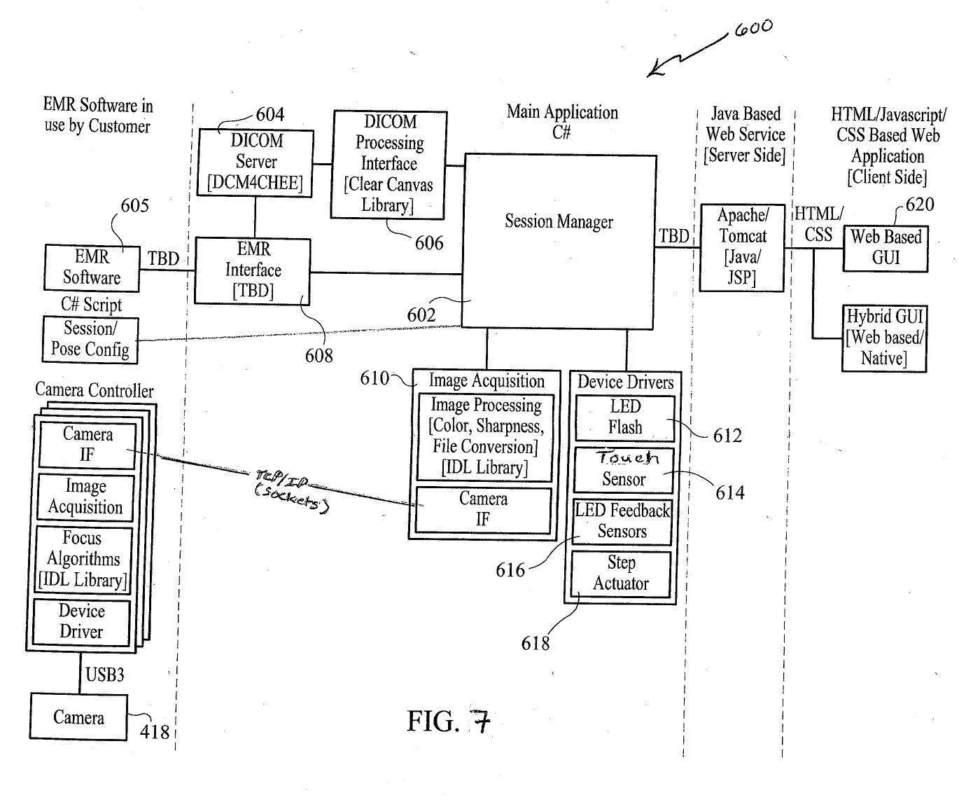

FIG. 7 is an exemplary software block diagram showing connections between the imaging station/device and other devices and interfaces that are included in the system for creating, processing, and displaying a total body image of a user. Software block diagram 600 includes a main application having a session manager 602 in communication with DICOM server 604 through a DICOM processing interface 606. The session manager 602 and DICOMserver 604 are also in communication with an Electronic Medical Records (EMR) interface 608 that is in turn in communication with EMR software 605 being used by medical practitioners and/or medical facilities. Session manager 602 is also in communication with image acquisition program 610 which includes a camera interface to cameras 418 and image processing abilities. Session manager 602 is also in communication with a device driver 612 for the light panels 422, a device driver 614 for touch sensors which may be located on the body positioning members 426 and a moveable step retractable within a floor or bottom of the imaging station/device that is used to assist in automatically capturing user/patient images, a device driver 616 for the LED feedback sensors associated with the body positioning members 426 and the movable step, and a device driver 618 for the step actuator associated with the moveable step. The session manager 602 is also in communication with a web based service that presents a web based graphical user interface (GUI) 620 to a medical professional/provider.

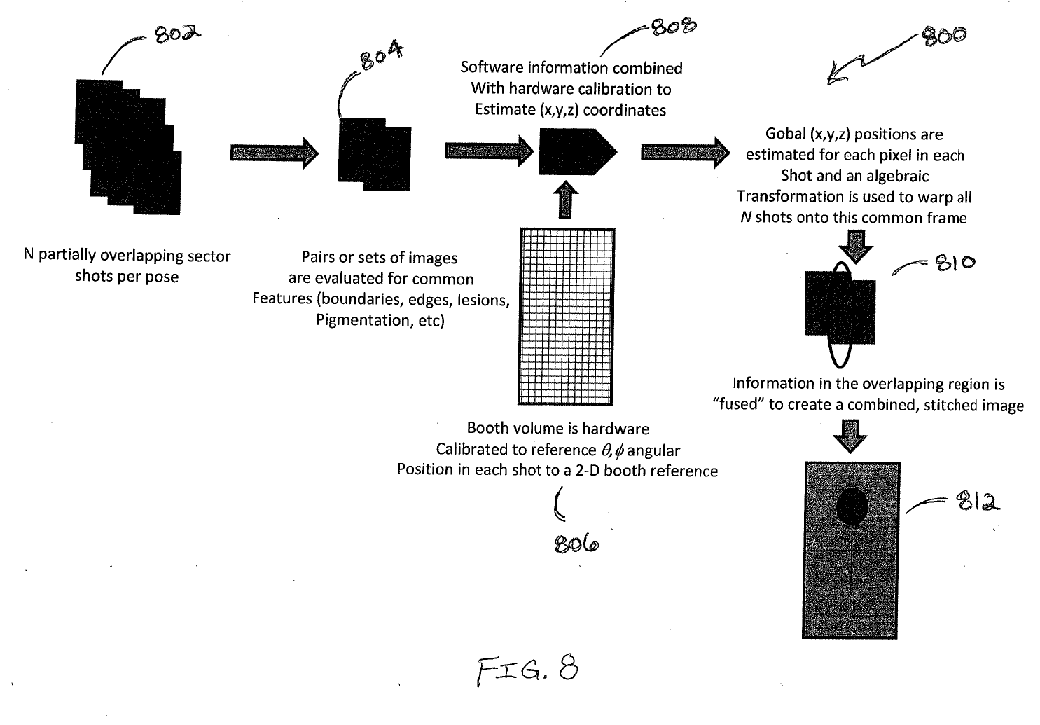

FIG. 8 is a schematic showing an exemplary process 800 of how the plurality of overlapping images of a user are stitched together to form a total body image of the user within the system for creating, processing, and displaying a total body image of a user. As shown in FIG. 8, the system and method for creating, processing, and displaying a total body image of the present invention includes taking a plurality (number N) of overlapping images of a user/patient per pose 802 that the user/patient is asked to undertake during an automated image taking session such as that done in accordance with using an imaging station/device such as those previously described and referenced. For example, an imaging station/device may have 9 total cameras where each camera captures an image of a specific portion (or sector) of the user/patient while the user/patient is in a particular pose. The number (N) of partially overlapping sector images are then evaluated in pairs or sets to determine common features 804 such as, for example, common boundaries, edges, lesions, pigmentation, etc. The volume of the imaging station/device used to capture the user/patient images is hardware calibrated 806 to reference specific angular positions in each image that is taken to a two dimensional booth reference. Once the common features of the overlapping images of a user/patient are identified, software information is combined with the hardware calibration 808 to estimate x,y,z coordinates. Global x,y,z positions are then estimated for each pixel in each image taken 810 and an algebraic transformation is used to wrap all N images onto a common frame. Finally, the information in the overlapping regions is “fused” together 812 to create a combined, stitched image.

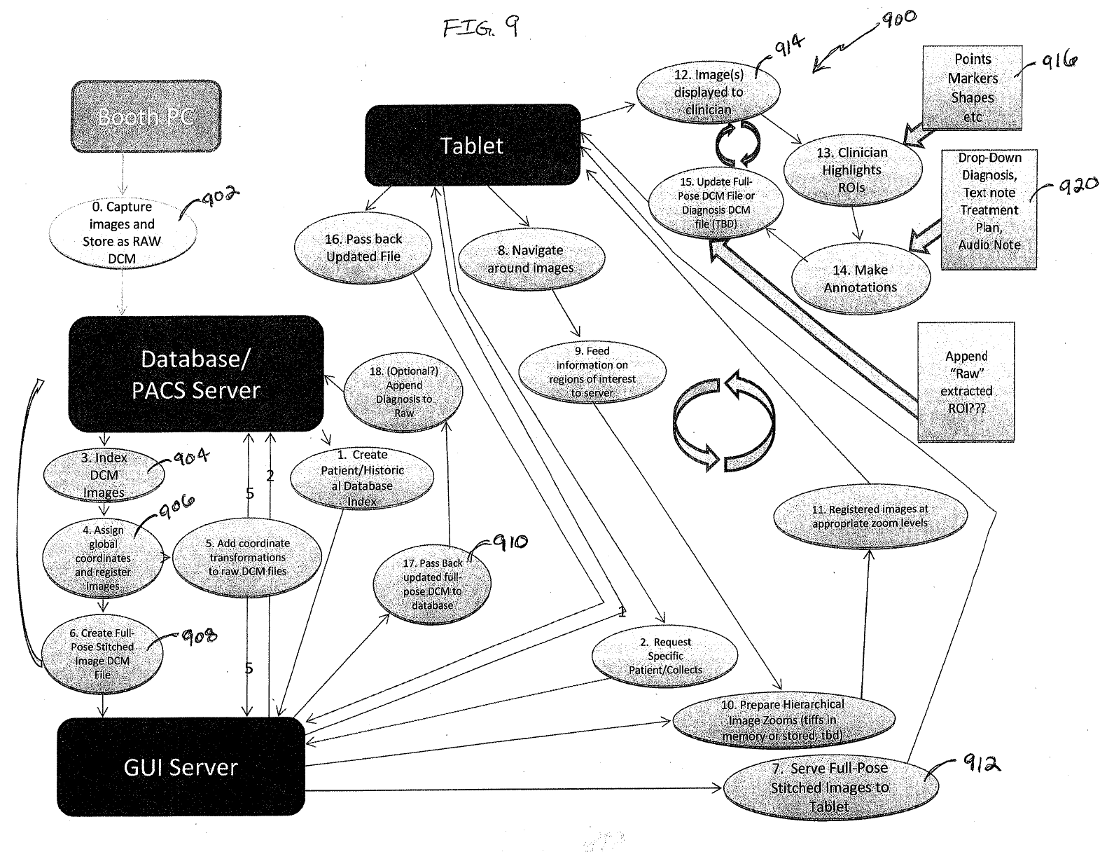

FIG. 9 is a flowchart showing an exemplary method 900 for creating, processing, and displaying a total body image of a user. As previously described, an imaging station/device/booth is used to capture images of a user/patient 902, the user/patient images are indexed 904, global coordinates are assigned and the images are registered 906, and a full posed or total body stitched image of the user/patient is created 908 The total body image of the user/patient is saved to a database 910 for future access. A medical professional/provider can access all images and/or the total body image of all users/patients that are saved to the system through a graphic user interface specifically designed for the medical professional/provider. Through a computer, an electronic notebook, an electronic tablet, or any other mobile computing device, a medical professional/provider can request specific user/patient information including user/patient images 912, the information and/or images including a total body image of the user/patient are then sent to the medical professional/provider, and the information and/or images including the total body image of the user/patient is displayed 914 to the medical professional/provider. Through the graphic user interface for the medical professional/provider, the medical professional/provider can make notations to the user/patient's file and can mark, highlight, etc. specific areas of concern 916 on the images of the user/patient and save those notations and markings for future reference when the medical professional/provider sees the user/patient again in the future. The graphic user interface can also, via drop down menu box or other means, enable a medical professional/provider to make a diagnosis, record written and/or audio notes and observations, create and document a treatment plan, and do a number of other actions relating to the management of a user/patient's medical care 920 including all of those actions previously described above in other parts of this specification. The hardware components used in carrying out these process steps are also shown in FIG. 9.

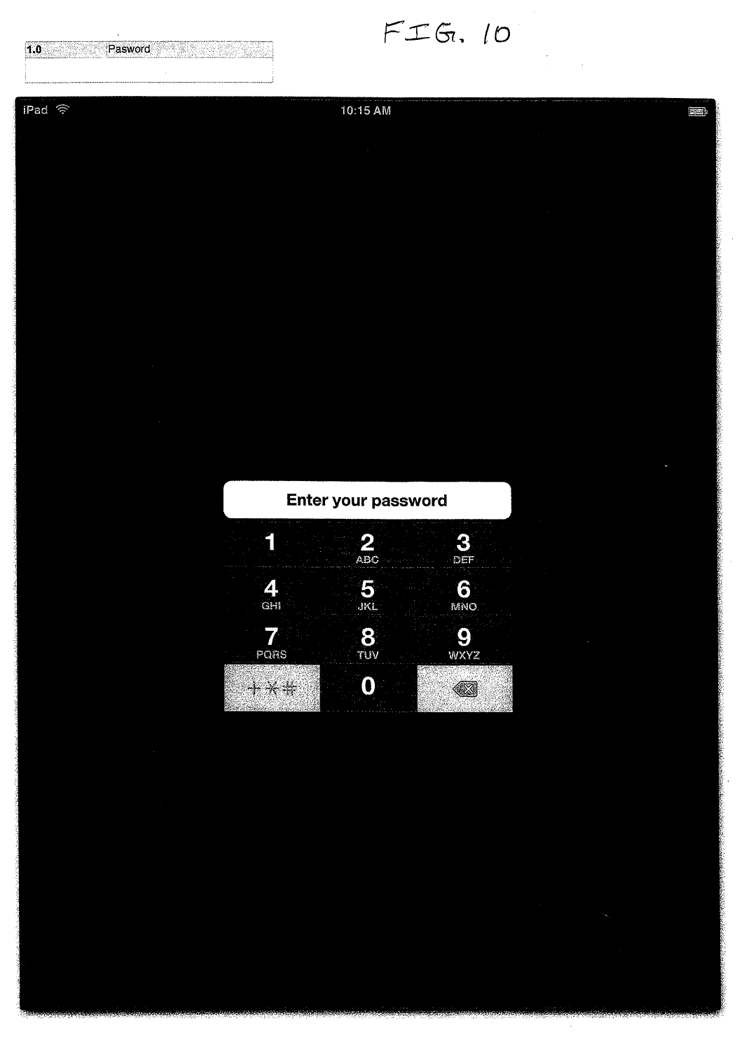

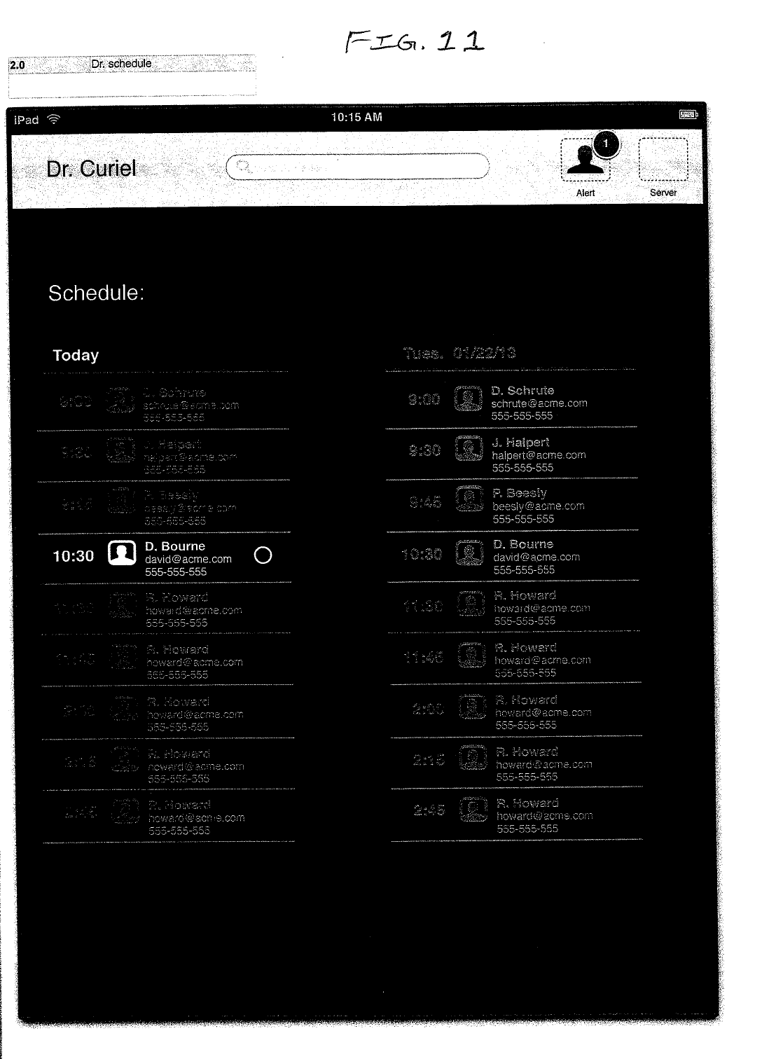

FIGS. 10-27 show exemplary frames/screen shots of the graphic user interface for medical professionals/providers used with the system and method for creating, processing, and displaying a total body image of a user in accordance with the present invention. FIG. 10 shows a screen shot requiring the input of a password by the medical professional/provider. Once the password is entered and accepted, the medical professional's/provider's schedule can be shown as depicted in FIG. 11. The medical professional/provider can then select the user/patient whose record they want to access, view, assess, and act on.

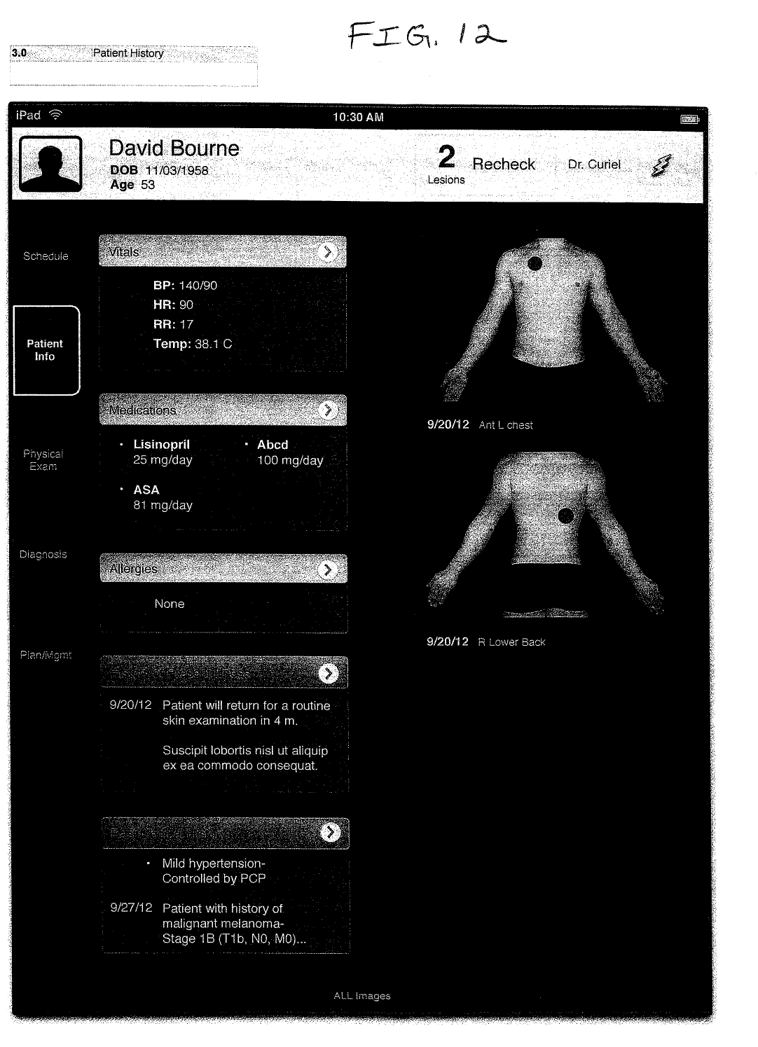

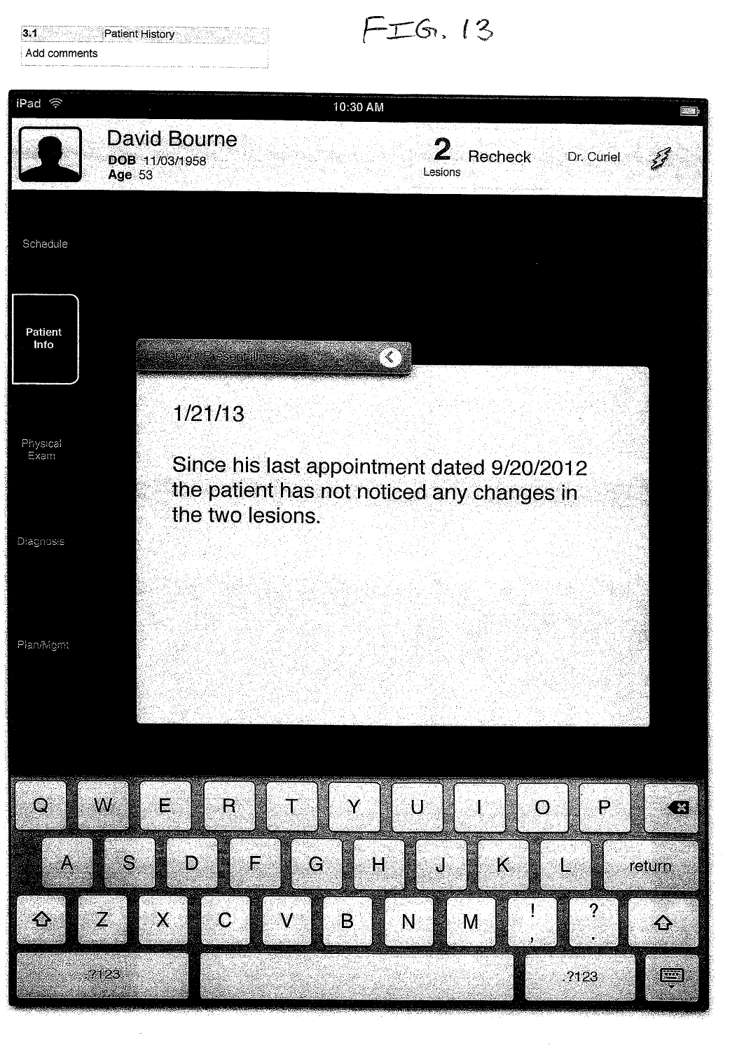

FIG. 12 shows a selected user/patient's info which includes vitals, medications, allergies, history of present illness, past medical history, and body images obtained from the system and method of the present invention for creating and processing a total body image. This screen also highlights to the medical professional/provider that 2 lesions on this user/patient need to be rechecked. In addition, this screen shows selections for this user/patient for schedule, physical exam, diagnosis, and plan/management—all of which can be selected and the used for viewing and input by the medical professional/provider. FIG. 13 shows an exemplary screen shot for the history of present illness for the user/patient.

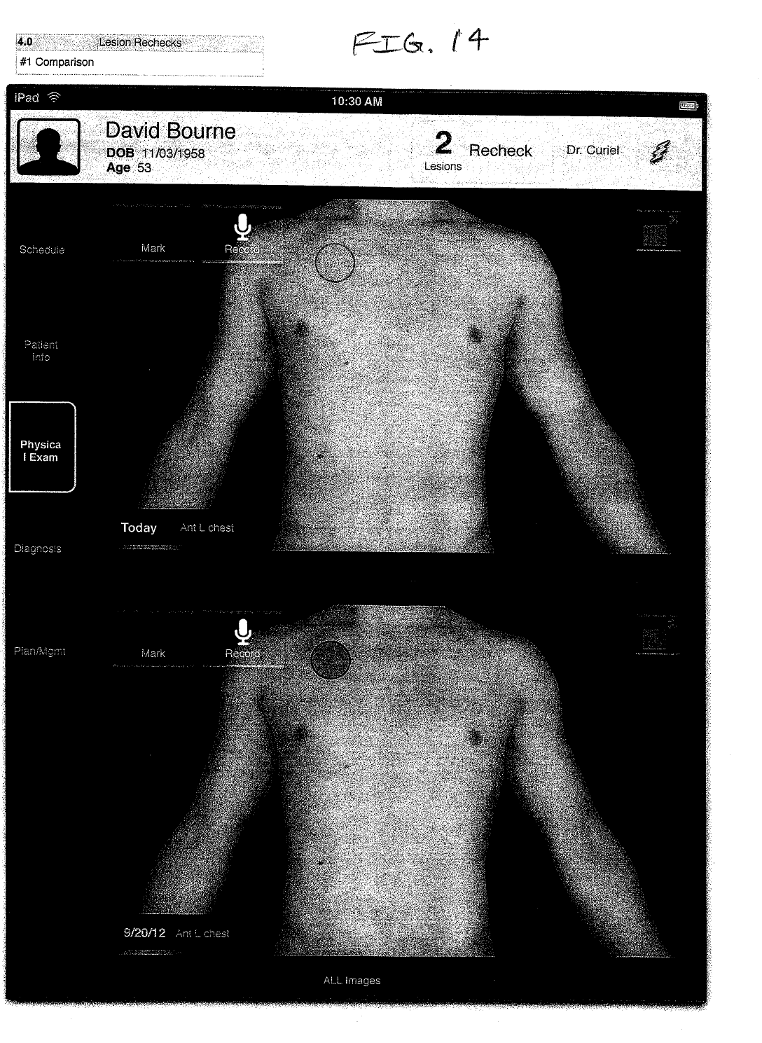

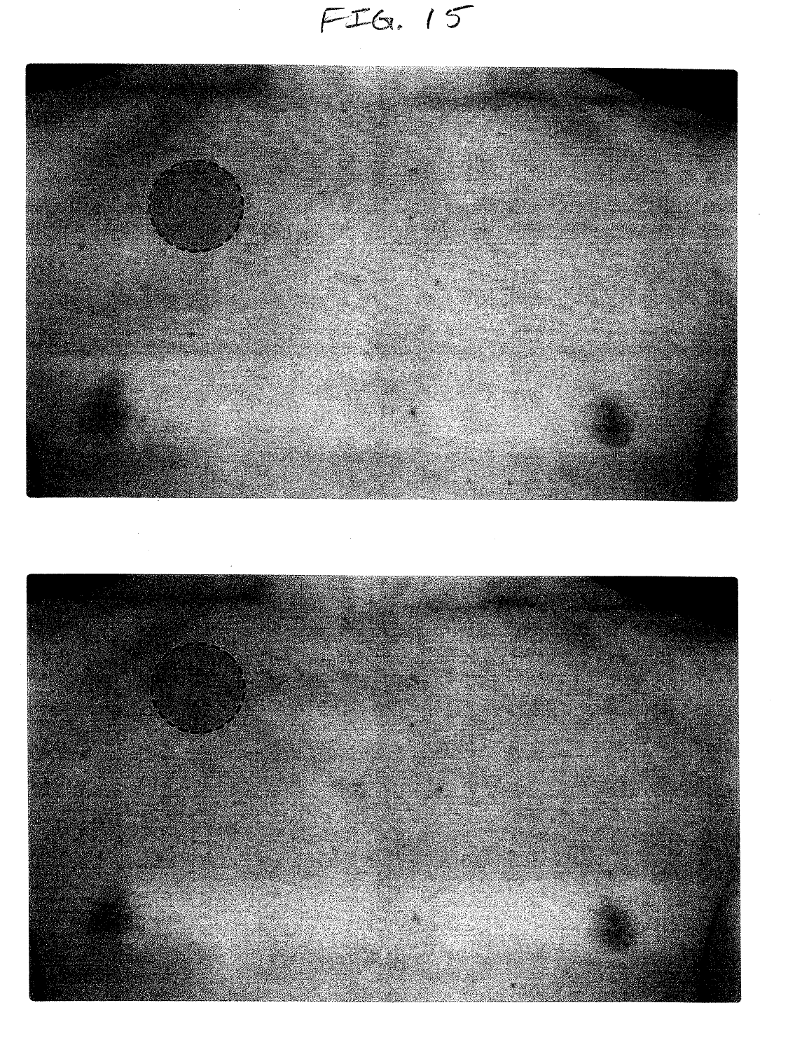

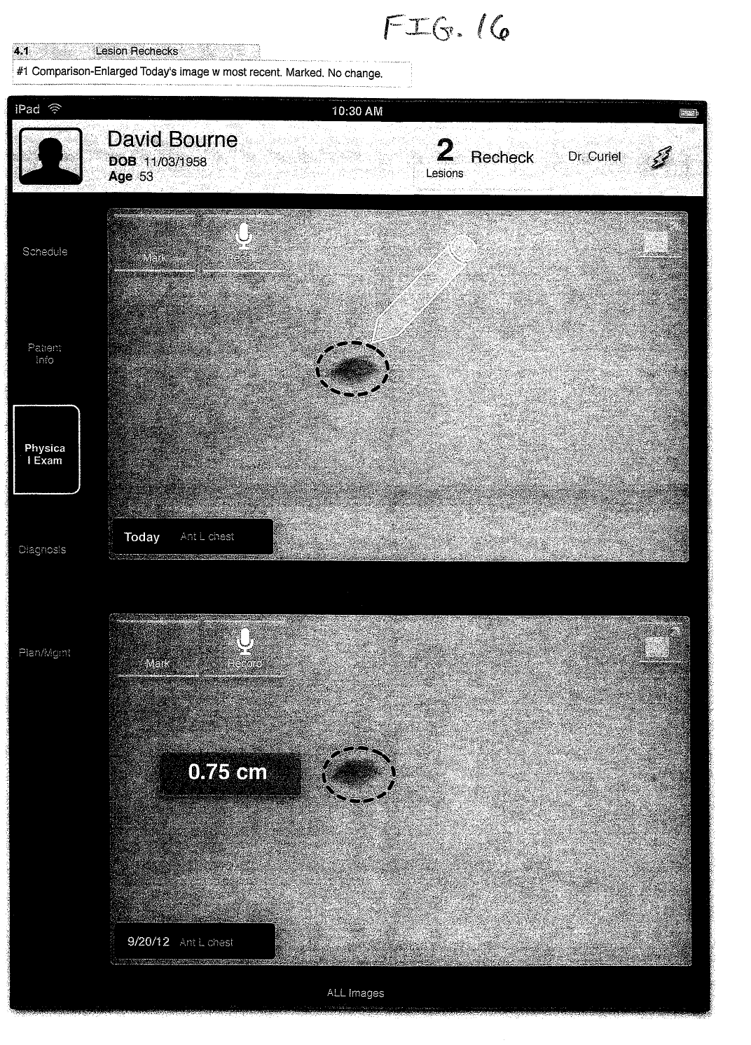

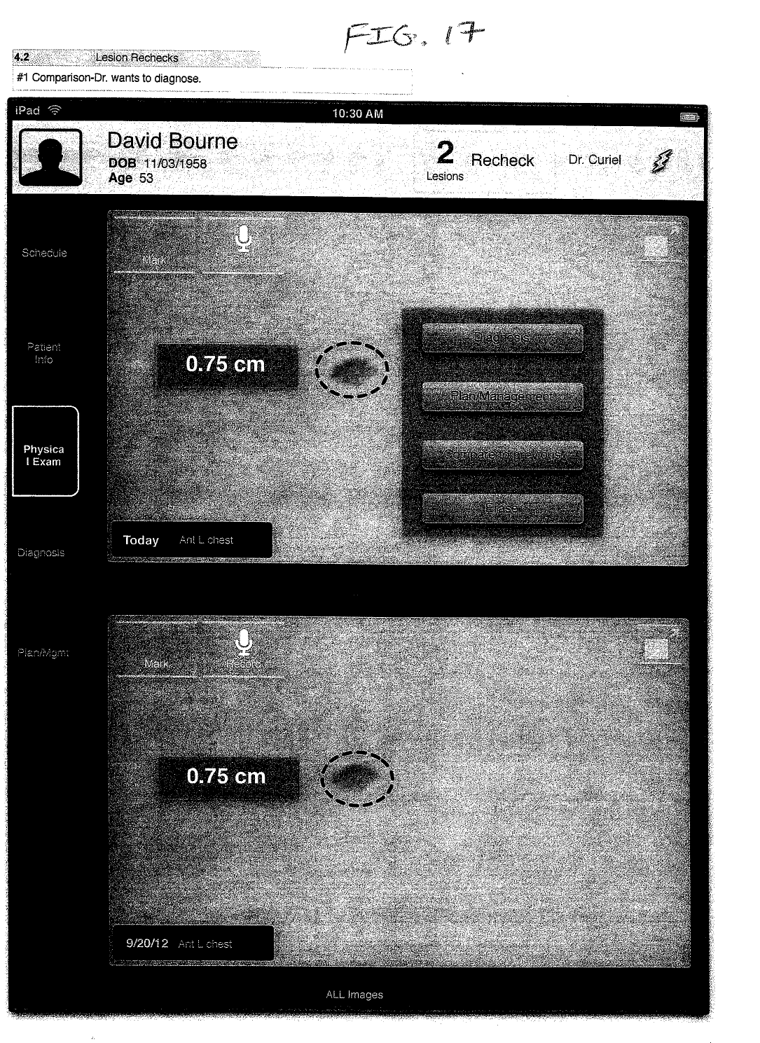

FIG. 14 shows a side by side (or tope and bottom) comparison of an image of the patient's lesion on anterior left chest taken at present (today) and several months ago. This is accessed under the physical exam option on the graphic user interface and the medical professional/provider can elect to mark the lesion with a labeling feature (see FIG. 16) and/or record specific observations about the lesion by audio recorder. FIG. 15 shows magnified views of the lesions shown in FIG. 14 as this ability to focus on and magnify specific abnormalities is one of the many options for the medical professional/provider using the graphic user interface. FIG. 17 shows an exemplary screen shot of a medical professional/provider's ability to mark and measure the lesion.

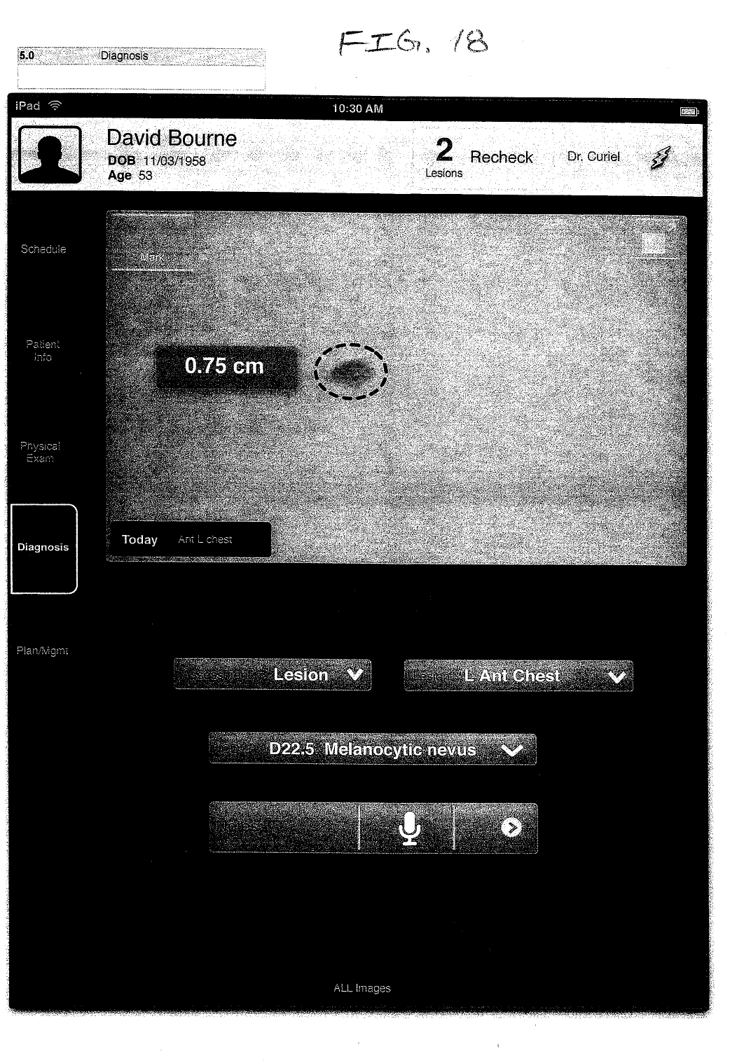

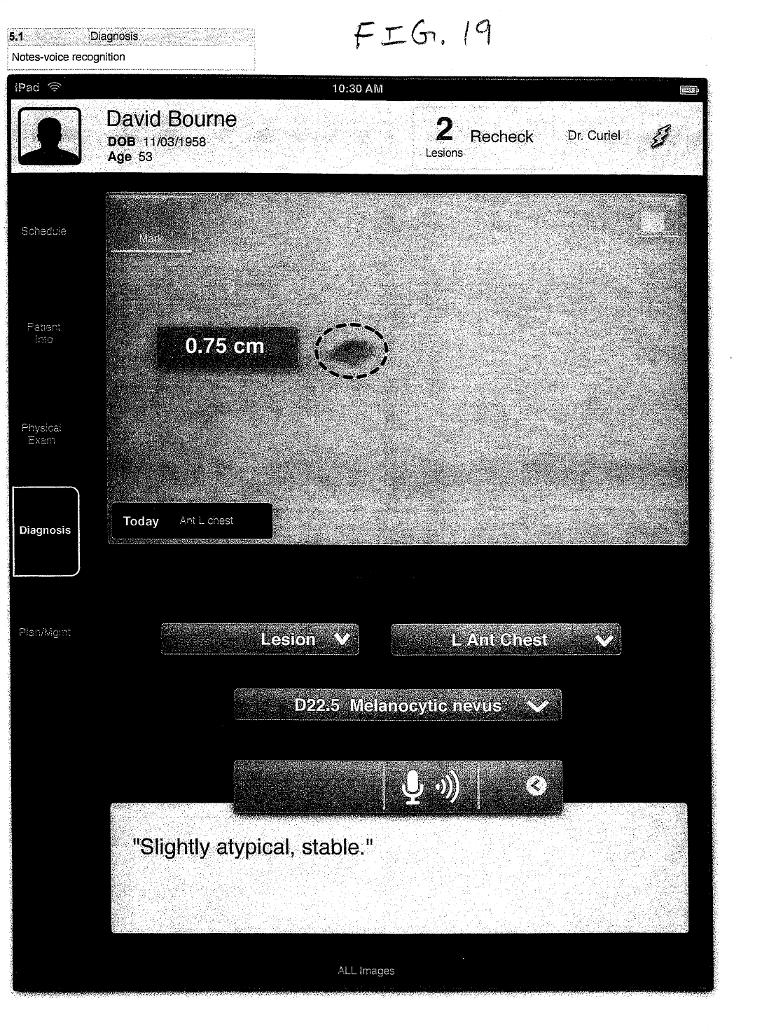

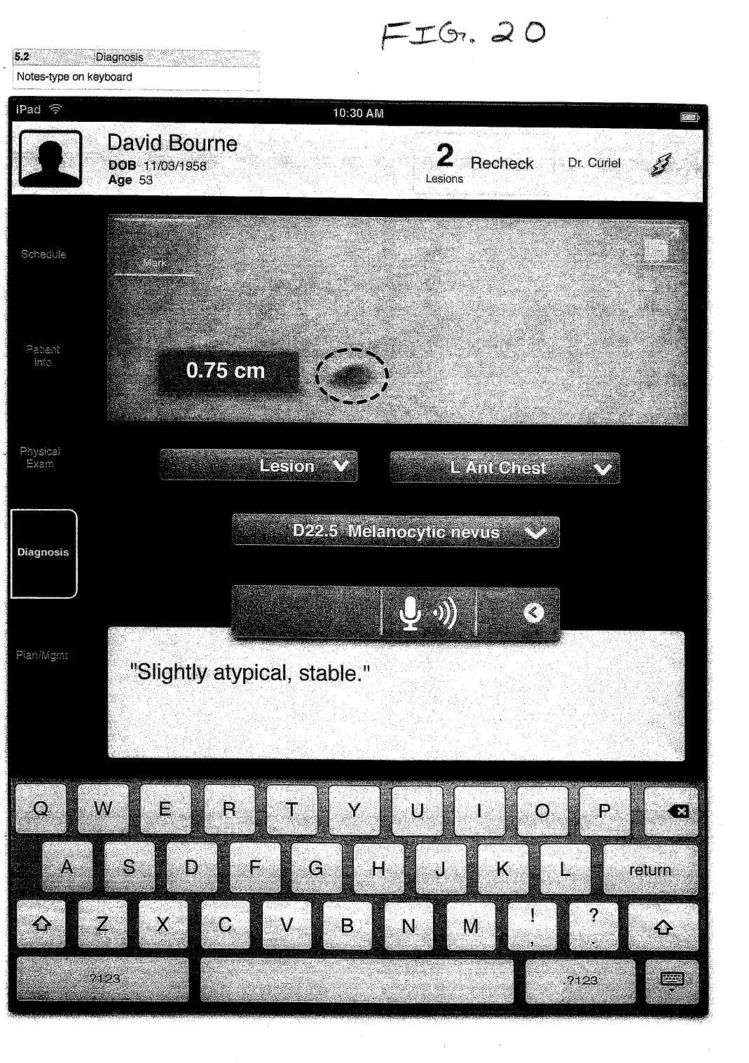

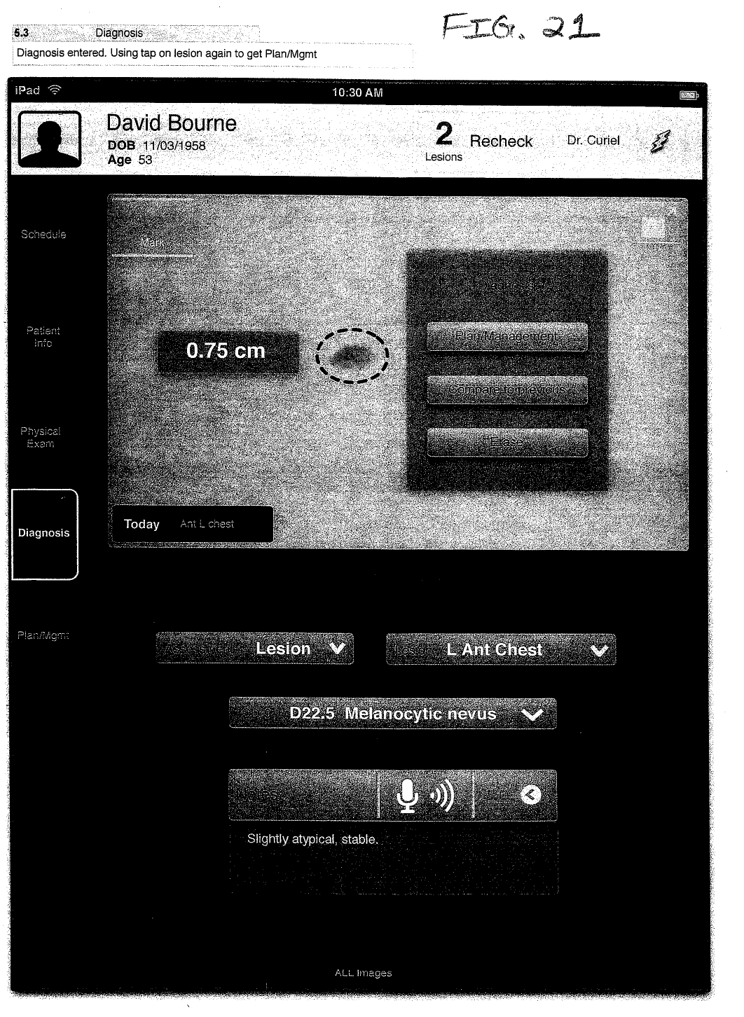

FIG. 17 shows a screen shot of a side by side comparison of the same lesion taken at different times (at present and months ago) and the size of the lesions at those times. FIG. 17 also shows the ability of the medical professional/provider to select various actions such as diagnosis, plan/management, compare to previous, and erase. FIG. 18 is a screen shot that shows the diagnosis feature of the graphic user interface with itemized areas on the screen for recording assessment of abnormality, abnormality location, medical related codes for the abnormality, and an option to provide written notes and/or record audio observations. FIG. 19 shows the same screen shot shown in FIG. 18 with the addition of an audio observation provided and entered by the medical professional/provider. FIG. 20 shows the same screen shot shown in FIG. 18 with a medical professional/provider providing a written observation that is entered by typing on a keyboard that can be hidden and/or accessed and FIG. 21 shows the written entry after being submitted and entered by the medical professional/provider.

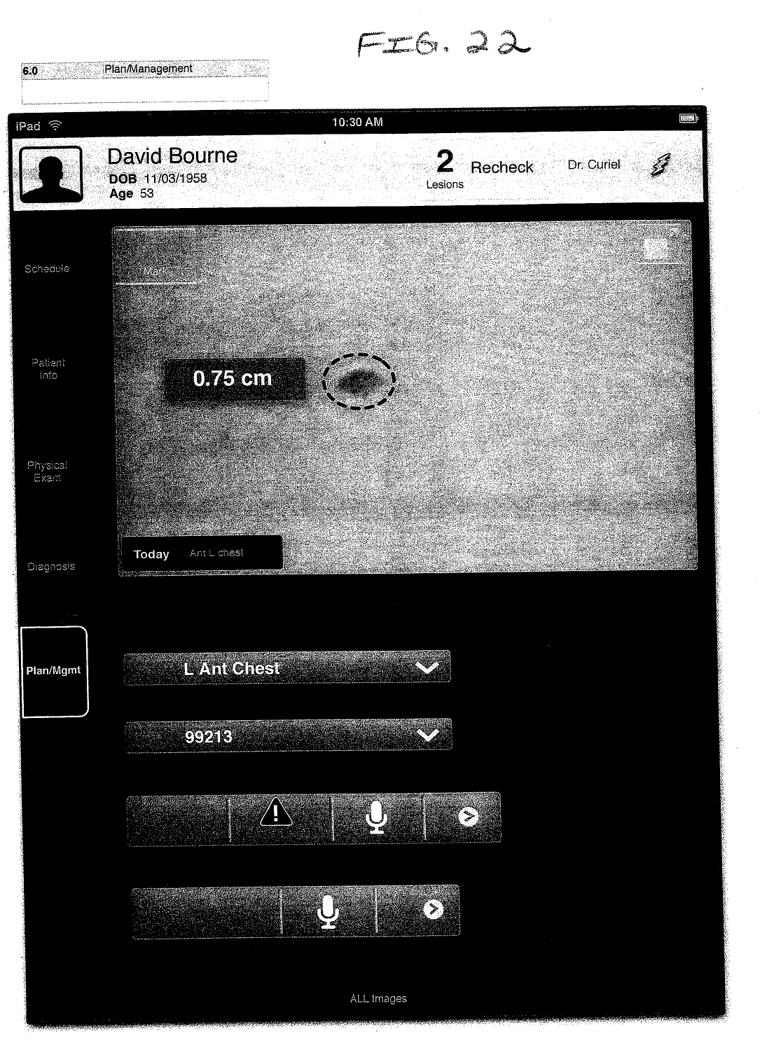

FIG. 22 is a screen shot that shows the plan/management feature of the graphic user interface with itemized areas on the screen for location of abnormality, medical codes relating to the abnormality, ability to set up an alert that an abnormality needs to be followed up on, and notes that can be entered via text or audio.

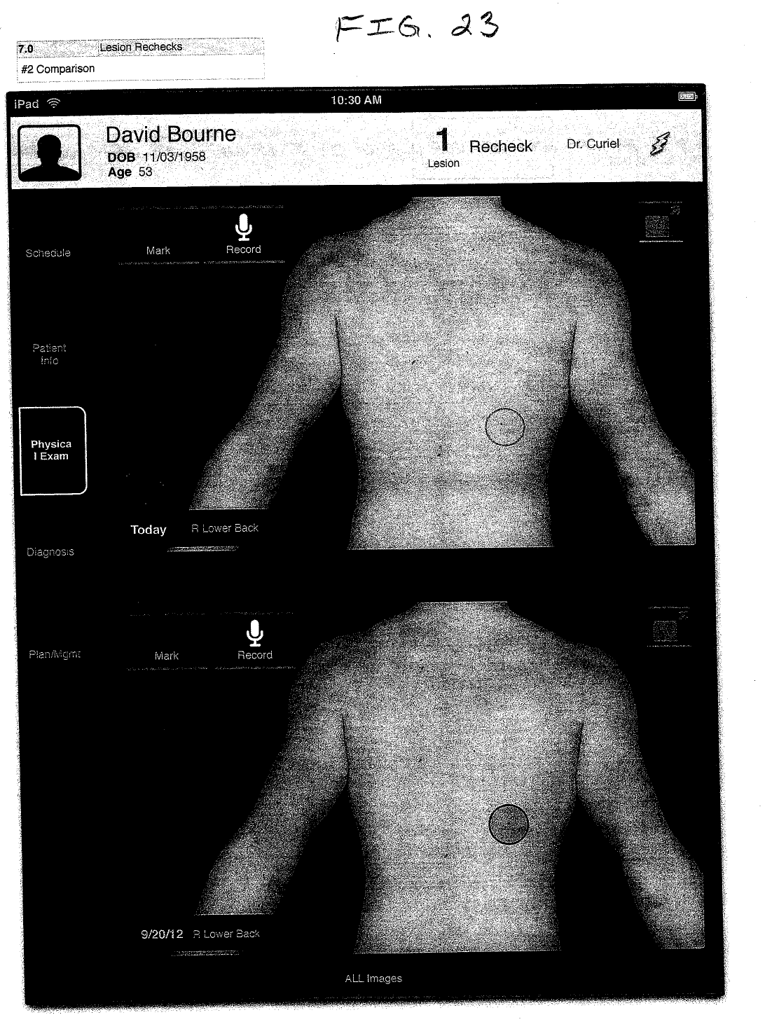

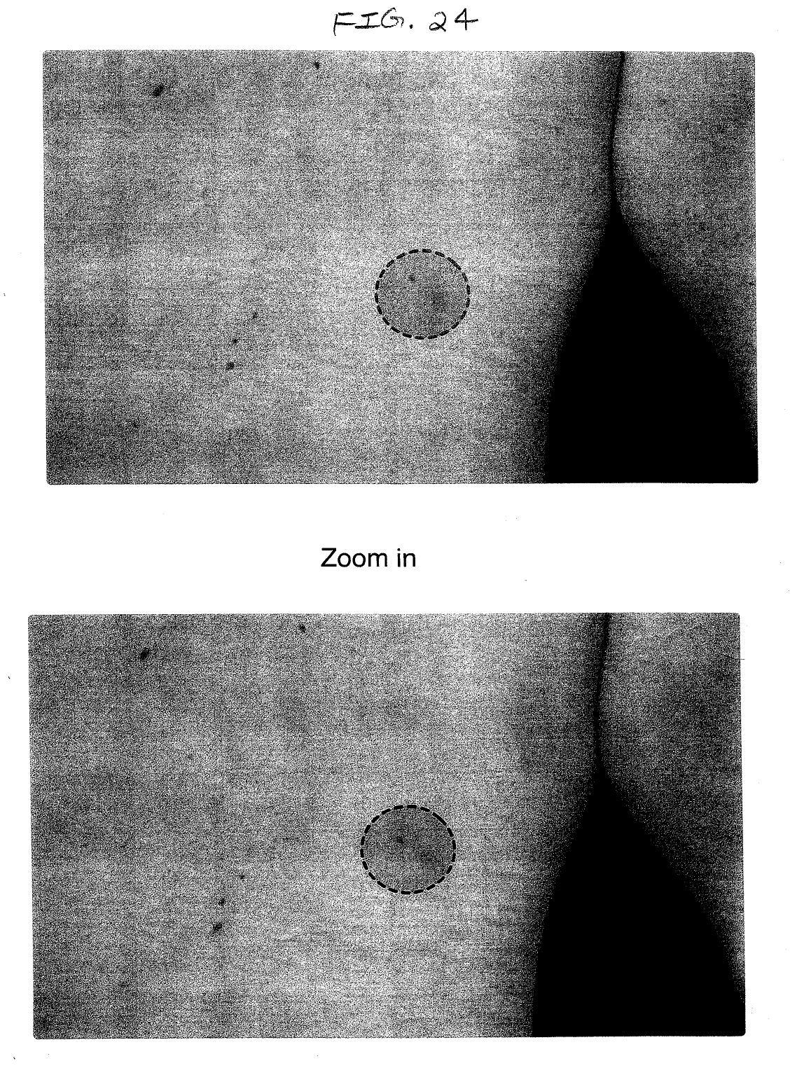

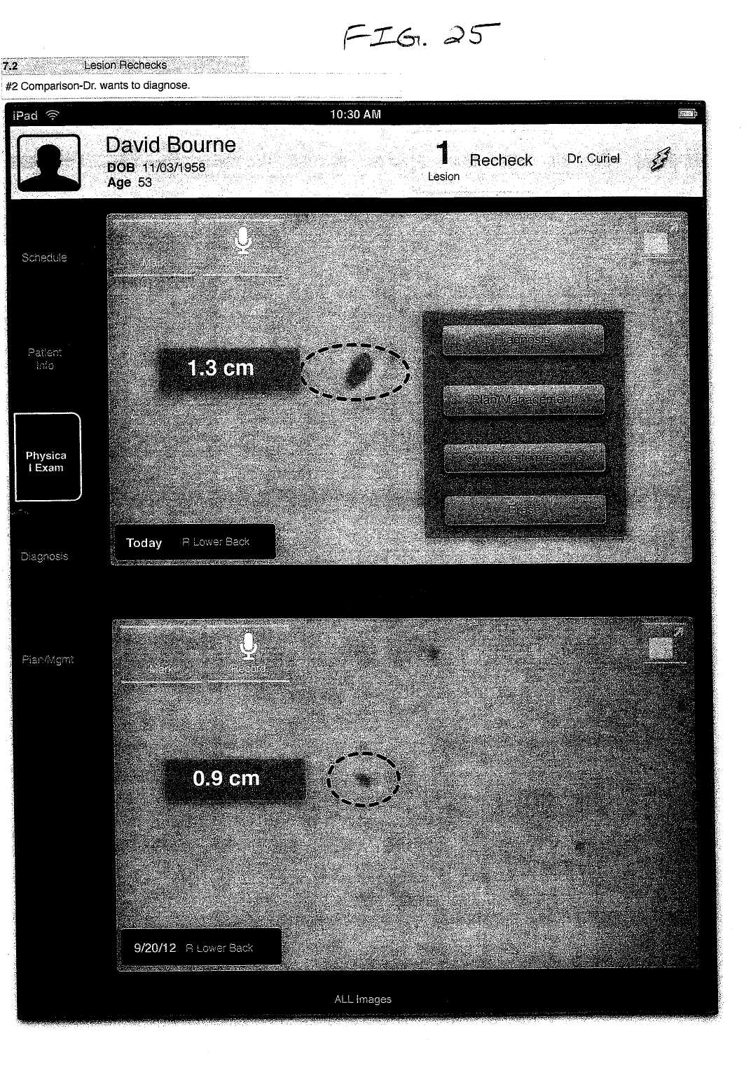

FIG. 23 shows a side by side (or tope and bottom) comparison of an image of the patient's lesion on his right lower back taken at present (today) and several months ago. This is accessed under the physical exam option on the graphic user interface and the medical professional/provider can elect to mark the lesion with a labeling feature (see FIG. 25) and/or record specific observations about the lesion by audio recorder. FIG. 24 shows magnified views of the lesions shown in FIG. 23 as this ability to focus on and magnify specific abnormalities is one of the many options for the medical professional/provider using the graphic user interface. FIG. 25 shows an exemplary screen shot of a medical professional/provider's ability to mark and measure the lesion.

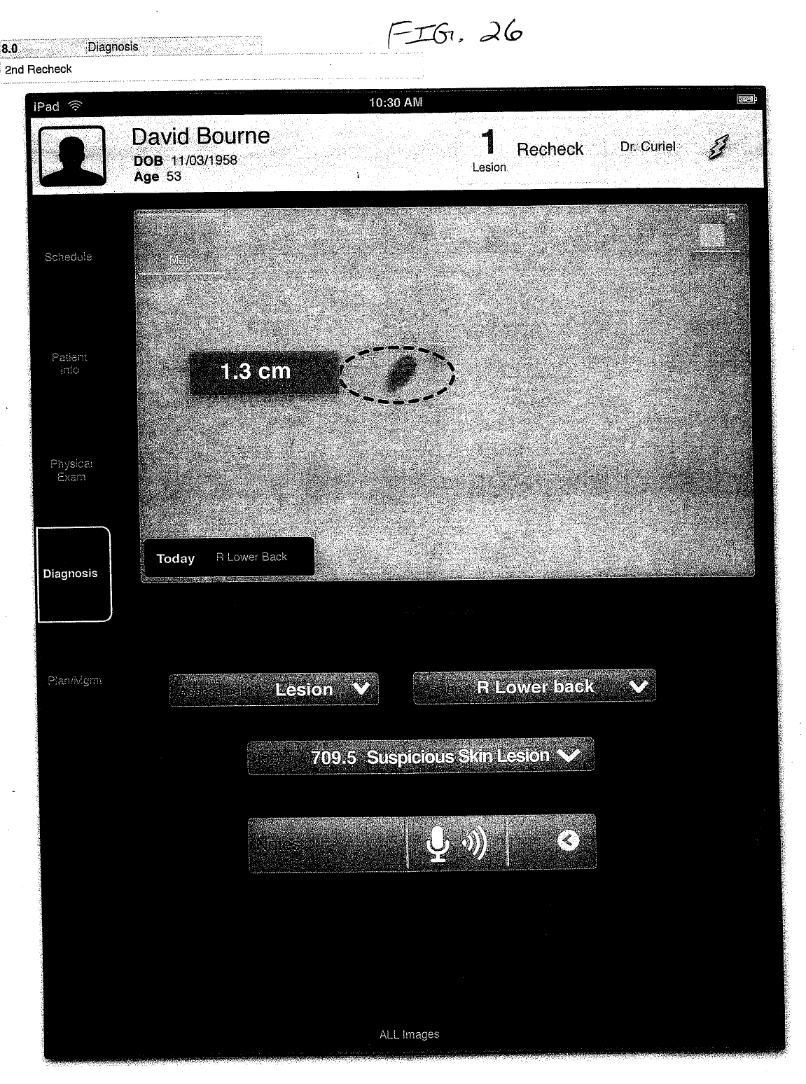

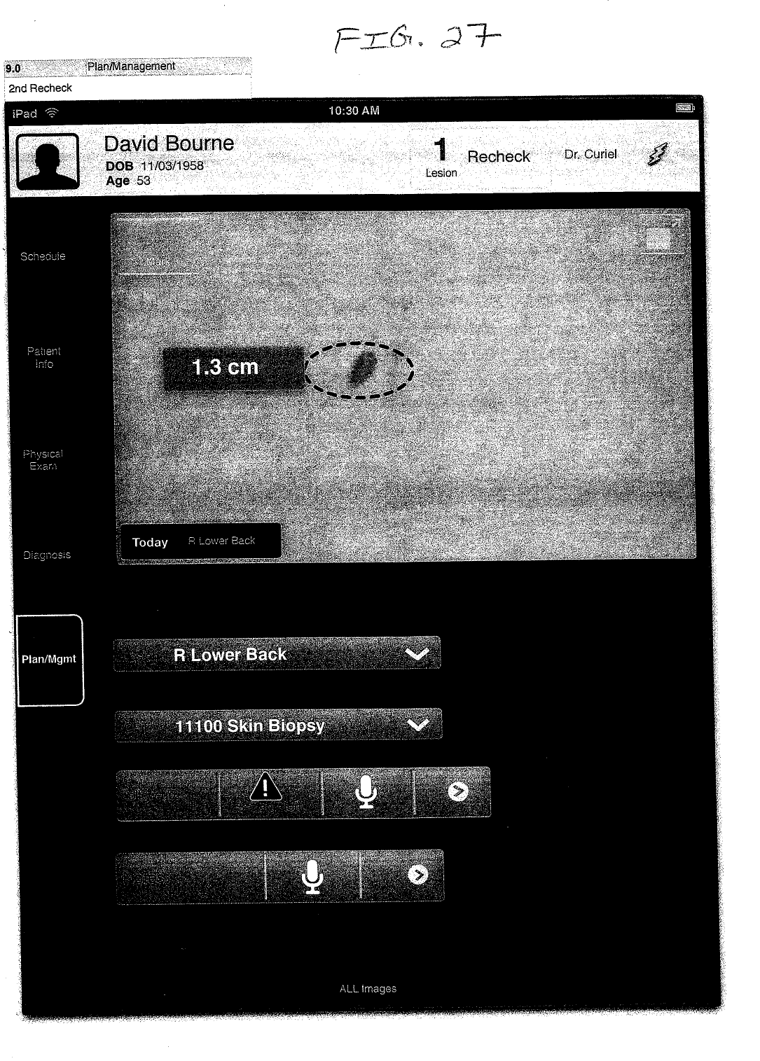

FIG. 26 is a screen shot that shows the diagnosis feature of the graphic user interface with itemized areas on the screen, relating to the present images in FIGS. 23-25, for recording assessment of abnormality, abnormality location, medical related codes for the abnormality, and an option to provide written notes and/or record audio observations. FIG. 27 is a screen shot that shows the plan/management feature of the graphic user interface with itemized areas on the screen for location of abnormality, medical codes relating to the abnormality, ability to set up an alert that an abnormality needs to be followed up on, and notes that can be entered via text or audio.

It will be understood by those skilled in the art that FIGS. 10-17 represent exemplary screen shots for the actions that can be taken by a medical professional/provider via the graphic user interface of the present invention for the medical professional/provider.

The present invention also contemplates enabling a technician/medical assistant and/or a medical professional/provider to bill users/patients for the imaging services and/or medical professional services they receive as they are incurred through the respective graphic user interfaces for the technician/medical assistant and the medical professional/provider.

The detailed description of exemplary embodiments of the invention herein shows various exemplary embodiments and the best modes, known to the inventor at this time, of the invention. These exemplary embodiments and modes are described in sufficient detail to enable those skilled in the art to practice the invention and are not intended to limit the scope, applicability, or configuration of the invention in any way. Rather, the following disclosure is intended to teach both the implementation of the exemplary embodiments and modes and any equivalent modes or embodiments that are known or obvious to those reasonably skilled in the art. Additionally, all included figures are non-limiting illustrations of the exemplary embodiments and modes, which similarly avail themselves to any equivalent modes or embodiments that are known or obvious to those reasonably skilled in the art.

Unless specifically noted, it is the Applicant's intent that the words and phrases in the specification and the claims be given the commonly accepted generic meaning or an ordinary and accustomed meaning used by those of ordinary skill in the applicable arts. In the instance where these meanings differ, the words and phrases in the specification and the claims should be given the broadest possible, generic meaning. If any other special meaning is intended for any word or phrase, the specification will clearly state and define the special meaning. It should be noted that the terms “imaging station”, “imaging device” and “imaging booth” are used interchangeably throughout and all three terms are not meant to be limiting. In addition is should be noted that “medical professional” and “medical provider” and “clinician” are used interchangeably throughout and are not meant to be limiting in any way.

1. A system for creating, processing, and displaying a total body image comprising:
a device for capturing a plurality of overlapping images of a user;
a server for storing and transmitting user images and user information; and
a processor in communication with a program application for stitching together the plurality of overlapping images of the user to create a total body image of the user.

2. The system of claim 1 further comprising:
at least one of a computer and/or a mobile computing device in communication with the server; and
a program application for a graphic user interface that enables a medical professional/provider to access user images and manage medical care for the user.

3. The system of claim 3 wherein said at least one of a personal computer, a tablet, or any other mobile computing device is in wireless communication with the server via a network.

4. The system of claim 1 wherein the server is capable of storing and transmitting user images and user information between entities.

5. The system of claim 1 wherein the device for capturing a plurality of overlapping images is automated.

6. The system of claim 5 wherein the automated device for capturing a plurality of overlapping images includes a customized auto focus algorithm that automatically adjusts the focus of one or more cameras for different user poses based on a desired body focus feature.

7. The system of claim 1 wherein the device for capturing a plurality of overlapping images includes the ability to control at least one of a retractable step contained within a floor of the device, a video display for users located within the device, an audio component for users located within the device, and a lighting element located at least partially within the device.

8. The system of claim 1 further comprising a network in communication with the server.

9. The system of claim 8 further comprising one or more external databases in communication with the server via the network.

10. The system of claim 2 wherein the program application for a graphic user interface that enables a medical professional/provider to access user images and manage medical care for the user includes a program application that enables a medical professional/provider to perform at least one of measuring and marking a skin abnormality with a labeling tool, recording text and/or verbal observations of the skin abnormality, comparing user images and/or user observations and/or data to previous same area user images and/or user observations and/or data taken and/or recorded at different times, recording diagnoses, recording and/or scheduling tests to be performed, creating alerts for the skin abnormality that it needs follow up, recording treatment plans and/or case management, and applying ICD (International Statistical Classification of Diseases and Related Health Problems) codes and/or CPT (Current Procedural Terminology) codes when diagnosing and/or assessing the sin abnormality using the graphic user interface for medical professionals/providers.

11. A method for creating and processing a total body image comprising the steps of:
capturing a plurality of overlapping images of a user; and
stitching together the plurality of overlapping images of the user to create a total body image of the user.

12. The method of claim 11 wherein the step of capturing a plurality of overlapping images of a user includes the step of capturing a plurality of overlapping images utilizing an automated imaging station/device.

13. The method of claim 12 further comprising the step of providing a graphic user interface for a technician/medical assistant that enables the technician/medical assistant to control one or more aspects of capturing the plurality of overlapping images of the user utilizing the automated imaging station/device.

14. The method of claim 12 further comprising the step of utilizing an automatic focusing algorithm to automate focusing of cameras within the imaging station/device by determining an area of interest for each camera in each of one or more predetermined body poses undertaken by a user.

15. The method of claim 11 further comprising the step of accessing a server to store and transmit user images and user information.

16. The method of claim 15 further comprising the step of transmitting the user images and user information to at least one of a computer and/or a mobile computing device utilizing a graphic user interface that enables a medical professional/provider to access the user images and manage medical care for the user.

17. The method of claim 16 wherein the medical professional/provider that accesses the user images is able to focus on and magnify areas within the total body image of the user/patient that are selected by the medical professional/provider.

18. The method of claim 16 wherein the medical professional/provider that accesses the user images is able perform at least one of measuring and marking a skin abnormality with a labeling tool, recording text and/or verbal observations of the skin abnormality, comparing user images and/or user observations and/or data to previous same area user images and/or user observations and/or data taken and/or recorded at different times, recording diagnoses, recording and/or scheduling tests to be performed, creating alerts for the skin abnormality that it needs follow up, recording treatment plans and/or case management, and applying ICD (International Statistical Classification of Diseases and Related Health Problems) codes and/or CPT (Current Procedural Terminology) codes when diagnosing and/or assessing the sin abnormality using the graphic user interface for medical professionals/providers.
